(19) United States
(12) Patent Application Publication
(10) Pub. No.: US 2024/0304325 A1
(43) Pub. Date: Sep. 12, 2024

(54) Static Multiomic Seed Approach for Identifying Molecular Signatures

(71) Applicant: Merck Sharp & Dohme LLC, Rahway, NJ (US)

(72) Inventor: Gunjan Singh Thakur, Rahway, NJ (US)

(21) Appl. No.: 18/118,734

(22) Filed: Mar. 7, 2023

(51) Int. Cl. G16H 50/20; G16H 70/60

(52) U.S. Cl. CPC G16H 50/20; G16H 70/60

(57) ABSTRACT

Disclosed is a method for obtaining a dataset containing data values for a plurality of parameters corresponding to biological factors. Seed features that are known to be correlated to a phenotype are selected from the plurality of parameters. A seed correlation network is constructed indicating pairs of correlated seed features. Candidate nodes correlated to the pairs of seed features are added to the seed correlation network. Paths are identified in the seed correlation network between a pair of seed features. Corresponding predictive powers for each path in the plurality of paths are determined that indicated the likelihood that the path correctly predicts a vaccination response. Paths are discarded that have a predictive power less than a predictive power of the pair of seed nodes. The remaining paths are ranked based on their predictive powers, providing biological insight into probable paths involved in a vaccination reaction.

### BACKGROUND

### 1. Technical Field

This disclosure relates generally to determining the biological pathways involved in a phenotype and, in particular, to using a machine learning model to identify interrelated risk factors for vaccine responses.

### 2. Background Information

Individuals may have a range of outcomes in response to vaccines. Some may respond well, developing large amounts of antibodies for fighting the target disease. Others may respond weakly, developing few or no helpful antibodies. Yet others may have a response that is an adverse reaction to receiving the vaccine. Currently, it is difficult to determine the biological reason for how any given individual responds to a vaccine. It is not always apparent what specific biological components, such as specific proteins, RNA sequences, or antibodies, are involved in the pathway of a vaccination response. How an individual responds to a vaccine is often dependent on complex biological interactions between multiple observables. Current practices may require several blood tests after receiving a vaccination, to assess changes in blood composition that could cause a vaccine response or lack thereof. Furthermore, even when a response is determined, the underlying reasons for that response remain difficult to understand.

### BRIEF DESCRIPTION OF THE DRAWINGS

Embodiments of the disclosure have other advantages and features which will be more readily apparent from the following detailed description and the appended claims, when taken in conjunction with the examples in the accompanying drawings, in which:

FIG. 1 is an environment diagram of an example system for predicting a response to vaccinations, in accordance with some embodiments.

FIG. 2 is a block diagram of a computing server for predicting a response to vaccinations, in accordance with some embodiments.

FIG. 3 is a flowchart of a method for ranking proposed paths corresponding to biological factors, in accordance with some embodiments.

FIG. 4 is a diagram illustrating paths between nodes in a multi-layer data scheme, in accordance with some embodiments.

FIG. 5 is a block diagram illustrating an example of a computer suitable for use in the system of FIG. 1, according to some embodiments.

### DETAILED DESCRIPTION OF THE PREFERRED EMBODIMENTS

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### Overview

The goal of administering a vaccination is typically to cause an increase in expression of a target antibody in a subject. In human patients, vaccinations cause an array of biological reactions. In some cases, patients may have a positive reaction of increased target antibody expression. However, the same vaccination may cause a negative reaction of decreased or no target antibody expression. Additionally, the same vaccination may cause a physical reaction similar to the symptoms of a cold, other temporary sickness, or worse.

It is difficult to assess the biological factors behind any one of the aforementioned vaccination responses. There may be a multitude of biological factors involved in a vaccination response. To understand and account for the role of each biological factor a clinician would have to perform a longitudinal multiomics profiling study. This requires multiple tissue samples from a recently vaccinated patient over set time intervals. Both the data generation and analysis is a challenging task, creating an excessive time demand to unravel the complex molecular mechanism of vaccine response. It is additionally challenging to parse between biological noise and signals corresponding to biological factors. Currently, state of the art machine learning methods fall short in detecting signals corresponding to biological factors against noise in a vaccination response. Therefore, a method for assessing biological pathways and factors involved in a vaccination response is desirable.

In one embodiment, a method includes obtaining a multiomics dataset divided into layers. In some embodiments, the layers of data are subsets of the dataset divided based on a time that a corresponding tissue sample is taken. Each layer of data represents a different omics data which encapsulates a plurality of biological parameters. These parameters correspond to biological factors collected during different time periods, for example, a protein layer, an RNA layer, an antibodies layer, a white blood cell layer, a red blood cell layer, a cytokine layer, or an adjuvant layer. Seed features are defined as features that are correlated to the phenotype (i.e, the vaccine response status: a categorical variable). The method constructs a correlation network among seed features. Further, the correlation network is expanded by adding features, defined as candidate nodes, that are correlated to a pairs of seed features. In addition, the method accounts for correlations between nodes across multiple time points. Emphasis is laid to the uni-directionality of edges between timepoints, as information can only flow from features at an earlier timepoint to features with a later timepoint. Selecting seed features may be based on a categorical question. The method includes identifying a plurality of paths in the correlation network between any pair of seed features. For each path identified, the method determines a corresponding predictive power. The corresponding predictive power indicates a likelihood of nodes in the path correctly predicting a vaccination response. In some embodiments, the predictive power of each path is calculated based on a product of an area under a curve and an error rate associated with the path. The method determines the area under the curve and the error rate associated with the path by fitting a random forest model to the path, in accordance with some embodiments. The method includes discarding one or more paths of the plurality of paths if their corresponding predictive power is less than a predictive power of the pair of seed nodes. The method further includes ranking each path in the plurality of paths based on the corresponding predictive power. The method ranks each path to provide insight into biological factors.

### Example System Architecture

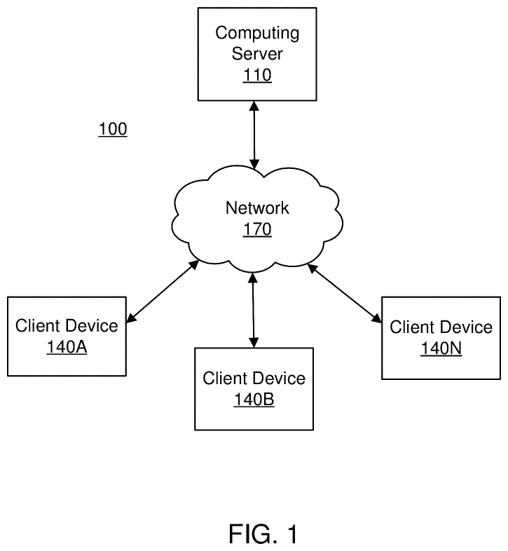

FIG. 1 illustrates one embodiment of a system 100 for assessing biological pathways potentially involved in a vaccination response. In the embodiment shown, the system 100 includes a computing server 110, network 170, and a plurality of client devices, 140A, 140B, and 140N. In other embodiments, the system 100 includes different or additional elements. For example, the system 100 could include any number of client devices. In addition, the functions may be distributed among the elements in a different manner than described.

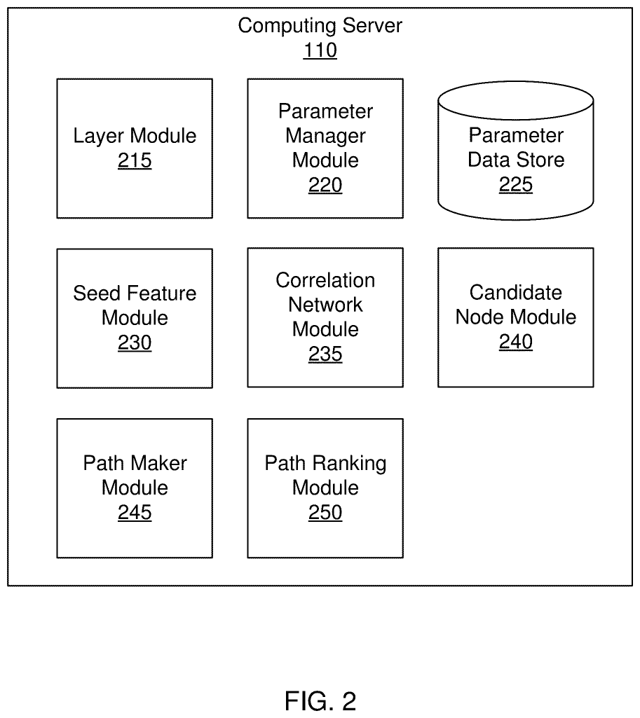

The computing server 110 is one or more computing devices that analyze biological data collected for subjects that have received a vaccination to identify combinations of biological factors that correlate with vaccine response. A client device 140 is a computing device that may be used to submit data to the computing server 110 or receive and display results from the computing server 110. In some embodiments, client device 140A is located at a health clinic. A clinician may administer a vaccination to a patient and take tissue samples of the patient at set time intervals. The tissue samples are run on an analysis machine which may provide results to the computing server 110 either directly or via the client device 140. The computing server 110 may provide results (e.g., a report) to the same or a different client device 140 indicating correlations between sets of biological factors and vaccine response. Various embodiments of the computing server 110 are described in greater detail below with reference to FIG. 2.

In some embodiments the analysis may be performed with a high-performance liquid chromatography (HPLC) machine, next generation sequencing for RNA sequencing data, mass spectroscopy for metabolomics, or another omic analysis approach. In some embodiments the analysis machine performs antibody titer tests. The clinician uploads the results of tissue sample analysis as a dataset to the network 170, in some embodiments. Each client device 140A, 140B, 140N, and possibly more client devices are used to provide a dataset containing information regarding tissue sample analyses from patients that received a vaccination to the network 170.

The network 170 provides the communication channels via which the other elements of the networked computing environment 100 communicate. The network 170 can include any combination of local area and wide area networks, using wired or wireless communication systems. In one embodiment, the network 170 uses standard communications technologies and protocols. For example, the network 170 can include communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 170 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 170 may be represented using any suitable format, such as hypertext markup language (HTML) or extensible markup language (XML). In some embodiments, some or all of the communication links of the network 170 may be encrypted using any suitable technique or techniques.

Referring to FIG. 2, the computing server 110 may contain a layer module 215, parameter manager module 220, parameter data store 225, seed feature module 230, correlation network module 235, candidate node module 240, path maker module 245, and path ranking module 250. The functions of the computer server 110 may be distributed among the elements in a different manner than described. In various embodiments, the computing server 110 may include different components and fewer or additional components. Each of the data stores may be a single storage device, a server controlling multiple storage devices, or a distributed network that is accessible through multiple nodes (e.g., a cloud storage system).

In some embodiments, the computing server 110 receives a tissue sample analysis dataset from the network 170. The layer module 215 of the computing server 110 divides the dataset into layers. Each layer includes data of a corresponding data type oromics dataset. In some embodiments, data of a corresponding data type may be data collected from a patient at a certain time. Each layer may include data values for a plurality of parameters. The parameters may correspond to multiple biological factors. The parameters corresponding to biological factors may be collected during different time periods, such as before administrating the vaccine, half an hour, an hour, a day, a week, and longer periods after administering a vaccination. The layer module 215 may create subsets of the dataset, called layers, based on each measurement time interval and data type. In some embodiments, the layer module 215 creates subsets of the dataset based on the parameters measured in the dataset. Each layer may correspond to multiple parameters measured at a given time.

In some embodiments, the parameter manager module 220 of the computing server 110 tracks measurements of each parameter in a plurality of parameters measured for patients. The parameter manager module 220 may identify parameters measured in the dataset. For example, the parameter manager module 220 can create parameter labels for proteins, RNA, antibodies, white blood cells, red blood cells, cytokines, and adjuvants. The parameter manager module 220 may manage which parameters to filter out of the dataset. For example, the parameter manager module 220 may filter out a protein data layer if the protein is determined to be an unrelated biological factor in a vaccination response. The parameter manager module 220 may use near zero variance to remove uninformative parameters, in some embodiments. The parameter manager module 220 may direct data for each parameter to the parameter data store 225. The computing server 110 may pull data for each parameter from the parameter data store 225 when analyzing biological pathways.

With continued reference to FIG. 2, the seed feature module 230 selects seed features from the plurality of parameters identified by the parameter manager module 220. The seed feature module may access the parameter data store 225 and select parameters known to be correlated to a phenotype. The seed feature module 230 performs feature selection in a supervised framework. Features may be selected based on being correlated to a phenotype. In some embodiments, the seed feature module 230 uses variable selection using random forests (VSURF), an ensemble-based method, to perform feature selection. VSURF or any other viable approaches to feature selection may be used. The seed feature module collates a set of features from all the data layers to produce a seed set.

A correlation network module 235 in the computing server 110 produces a correlation network. In one embodiment, the correlation network module 235 uses the seed set from the seed feature module 230 to construct the correlation network. The correlation network module 235 may calculate correlations between features in the seed set using Kendall's tau correlation coefficient. The correlation network module 235 may take steps to control for false positive rates in constructing the correlation network. In some embodiments, the correlation network module 235 sets a user-defined cutoff correlation value to determine whether an interaction is included in the correlation network. The correlation network module 235 may additionally create a global correlation network with all features from all the data layers. The correlation network module 235 identifies significant interactions in the correlation network by calculating a false discovery rate (FDR) corrected p-value and determining that the calculated p-value is less than a user-defined p-value.

The computing server 110 further contains a candidate node module 240. The candidate node module 240 grows the correlation network created by the correlation network module 235. The candidate node module 240 adds additional features to the correlation network created by the correlation network module 235. In one embodiment, the candidate node module 240 considers features that are strongly correlated to a pair of features in the seed set produced by the seed feature module 230. The features that are added by the candidate node module are referred to as candidate nodes. For a given pair of seed features, the candidate node module 240 identifies a candidate node that is strongly correlated to each of the seed features in the pair. The candidate node module 240 deems the candidate node to be strongly correlated with each of the seed features if the Kendall's tau correlation value between the candidate node and the seed features is above a user-defined threshold. The candidate node module 240 may additionally deem the candidate node to be strongly correlated with each of the seed features if the edges between each of the seed features and the candidate node are significant. In some embodiments, a signal to noise ratio in the dataset may be low. Depending on the feature selection approach used, the correlation network created by the correlation network module 225 may contain a sparse seed network, meaning that the number of edges in the network is small. The candidate node module 240 strategically increases the number of correlated features that are incorporated into the correlation network and chosen as candidate nodes. The candidate node module 240 correlates data across the layers of the dataset, accounting for biological crosstalk between the data layers.

The computing server 110 further includes a path maker module 245. The path maker module 245 identifies all paths between a pair of seed features in the seed set created by the seed feature module 230. In one embodiment, the path maker module 245 only considers paths of length greater than two and less than a user defined value.

A path ranking module 250 in the computing server 110 ranks paths from the path maker module 245. In one embodiment, to assess the predictive power of each path, the path maker fits a random forest (RF) model for each pair of seed features from the seed set produced by the seed feature module 230. The RF model training is cross validated (CV) and an area under the curve (AUC) and error rate (ER) are estimated. Performance metrics including the AUC and ER are averaged over a user-defined amount of CV loops. The path ranking module 250 extracts a top user-defined percentile of pairs of seed features based on their predictive power. The predictive power of each pair of seed features is calculated by multiplying the AUC and ER, in some embodiments. The path ranking module 250 discards of paths that are determined to be less than or equal to the predictive power of the seed pair alone, in some embodiments. In some embodiments, the path ranking module 250 produces a subnetwork with all the ranked paths. The path ranking module 250 may apply the Louvain community detection algorithm to identify clusters of heterogeneous markers and other network graph statistics. The Louvain community detection algorithm may determine node degree and centrality, as well, corresponding to one or more biological insights such as an enzymatic pathway or a biochemical reaction that may be involved in a vaccination reaction. Once generated, the path rankings may be used to guide further investigation into vaccine responses.

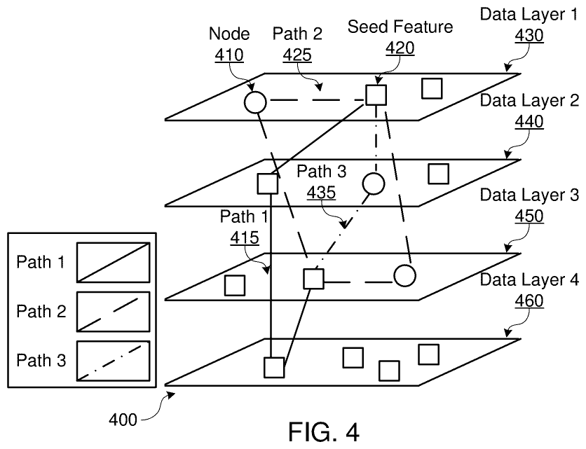

FIG. 4 illustrates an example architecture of a layered dataset for analysis by the computing server 110. The layer module 215 may divide a dataset into layers, depicted by data layer 1 430, data layer 2 440, data layer 3 450, and data layer 4 460. Each data layer may correspond to data taken from patients at a given time after administering a vaccination. Each data layer may contain a plurality of nodes 410 corresponding to parameters. Each data layer may additionally contain a plurality of seed features 420 that correspond to parameters. The path maker module 245 may generate a plurality of paths, illustrated by path 1 415, path 2 425, and path 3 435. As illustrated in FIG. 4, each generated path 415, 425, ad 435, connects two or more nodes and seed features. Each connected node or seed feature may represent a biological insight, such as a molecular response to a vaccination. By returning ranked paths with the path ranking module 250, the computing server 110 provides likely biological functions behind a vaccination response.

### Example Methods

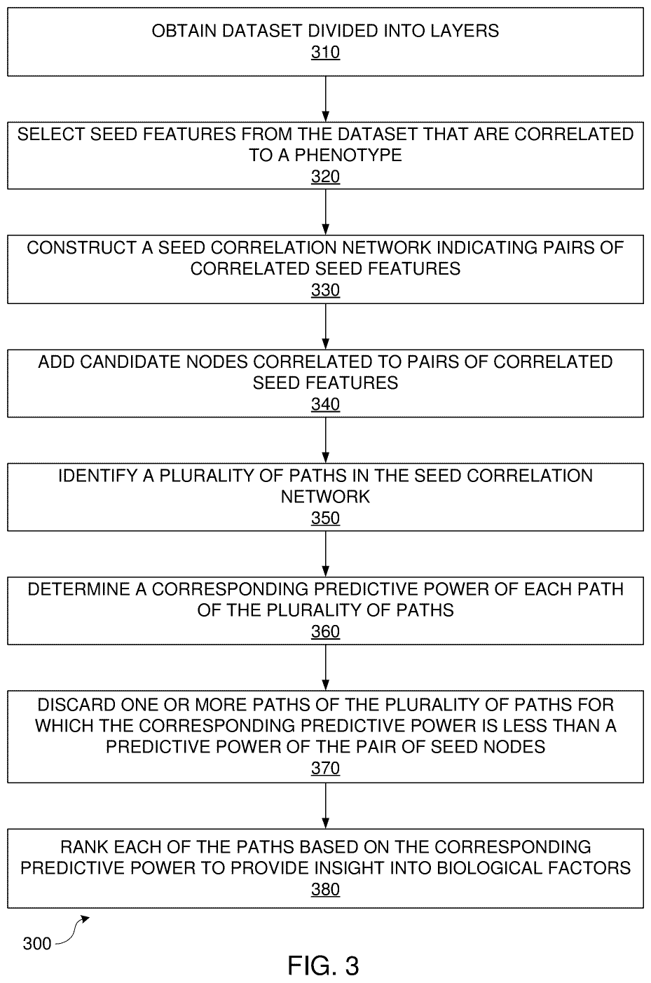

FIG. 3 illustrates a method 300 for determining potential biological factors involved in a vaccination reaction, according to one embodiment. The steps of FIG. 3 are illustrated from the perspective of the computing server 110 performing the method 300. However, some or all of the steps may be performed by other entities or components. In addition, some embodiments may perform the steps in parallel, perform the steps in different orders, or perform different steps.

In the method 300, the computing server 110 obtains a dataset divided into layers 310. The dataset may be uploaded to the computing server 110 through a network 170 from client devices 140A, 140B, 140N, etc. already divided into layers. For example, the clinician using client device 140A may divide data from a patient into layers based on the time of each corresponding blood sample. Alternatively, the layer module 215 divides a dataset from the client devices 140QA, 140B, 140N, etc. into layers based on parameter type. The layer module 215 may divide the dataset based on a time of blood sample analysis or blood sampling from a recently vaccinated patient. The layers obtained from a dataset divided into layers 310 include data of a corresponding data type. In some embodiments, at least some of the layers in the dataset include data values for a plurality of parameters. The parameters correspond to biological factors collected during different time periods. Biological factors may include at least one of proteins, RNA, antibodies, white blood cells, red blood cells, cytokines, and adjuvants. Other biological factors may be included that correspond to biochemical reactions or enzymatic pathways involved in a vaccine reaction phenotype. The parameter data store 225 of the computing server 110 tracks each parameter in the layers of the dataset.

With continued reference to the method 300 in FIG. 3, seed features are selected from the dataset that are correlated to a phenotype 320. The seed feature module 230 may apply a non-specific filtering technique such as near zero variance to remove uninformative features from the dataset. Uninformative features in the dataset may include features that remain static over time in the dataset or features that are determined not to be relevant to a vaccine response phenotype. In some embodiments, phenotypes include a positive response of increased target antibody production in a vaccinated patient. Phenotypes may include a negative or non-reaction of diminished or stagnant target antibody production in a vaccinated individual. Further, phenotypes may include a symptomatic reaction to a vaccination such as fever or other cold symptoms. The seed feature module 230 may then select seed features in a supervised framework. For example, the seed feature module may collect seed features most correlated to a phenotype. VSURF may be employed to select seed features from the dataset that are correlated to a phenotype 320.

A seed correlation network indicating pairs of correlated seed features is constructed 330. The correlation network module 235 of the computing server 110 constructs the seed correlation network. The seed correlation network may indicate pairs of seed features from a collated set of seed features from all the data layers. Correlations between features are computed by the correlation network module 235 using Kendall's tau correlation coefficient. Kendall's tau test is a non-parametric rank-based correlation method. Each layer in the dataset may have a different data distribution and noise structure, so the Kendall's tau test may account for these inconsistencies. The resulting seed correlation network from the correlation network module 235 comprises significant interactions above a threshold correlation value. In some embodiments, the threshold correlation value is user-defined.

Candidate nodes are added to the seed correlation network 340. Candidate nodes are correlated to pairs of correlated seed features. The candidate node module 240 considers additional features that are strongly correlated to a pair of features in the seed set. In some embodiments, determining that a feature is strongly correlated comprises determining that the Kendall's tau correlation value for a feature is above a user-defined threshold and that edges between the feature and each of the seed features in the pair is significant. For the edges between the feature and each of the seed features in the pair to be significant, the candidate node module 240 may determine that the edge has an FDR p-value below a user-defined threshold p value.

A plurality of paths are identified in the seed correlation network 350. The path maker module 245 generates a plurality of paths between a pair of correlated seed features. The paths generated by the path maker module 245 may represent a biochemical reaction, enzymatic pathway, or other interactions between biological factors. For each path identified in the seed correlation network 350, a corresponding predictive power is calculated 360. The predictive power indicates a likelihood of nodes in the path correctly predicting a vaccination response. The path ranking module 250 may calculate the predictive power of each path by fitting a RF model to the path. The path ranking module 250 cross validates the model and determines AUC and ER estimates. The product of the AUC and ER determines the predictive power of the path. A higher predictive power indicates an increased correlation between a path and a vaccination response.

The path ranking module 250 discards one or more paths of the plurality of paths for which the corresponding predictive power is less than a predictive power of the pair of seed nodes 370. The path ranking module 250 calculates the predictive power of the pair of seed nodes alone. The AUC and ER of the simplest path between the pair of seed nodes is determined as a threshold minimum predictive power. For paths produced by the path maker module 245, the path ranking module 250 discards any paths with a corresponding predictive power lower than that of the simplest path between the pair of seed nodes.

Each of the paths is ranked based on the corresponding predictive power to provide insight into biological factors 380. The path ranking module 250 uses calculated predictive powers to rank the paths based on their correlation to a vaccine reaction phenotype. A Louvain community detection algorithm may be applied by the path ranking module 250 to a user-defined percentile of top ranked paths to identify clusters of heterogeneous markers and other network graph statistics such as node degree and centrality. Information obtained from the Louvain community detection algorithm may be used to obtain biological insights.

### Computing System Architecture

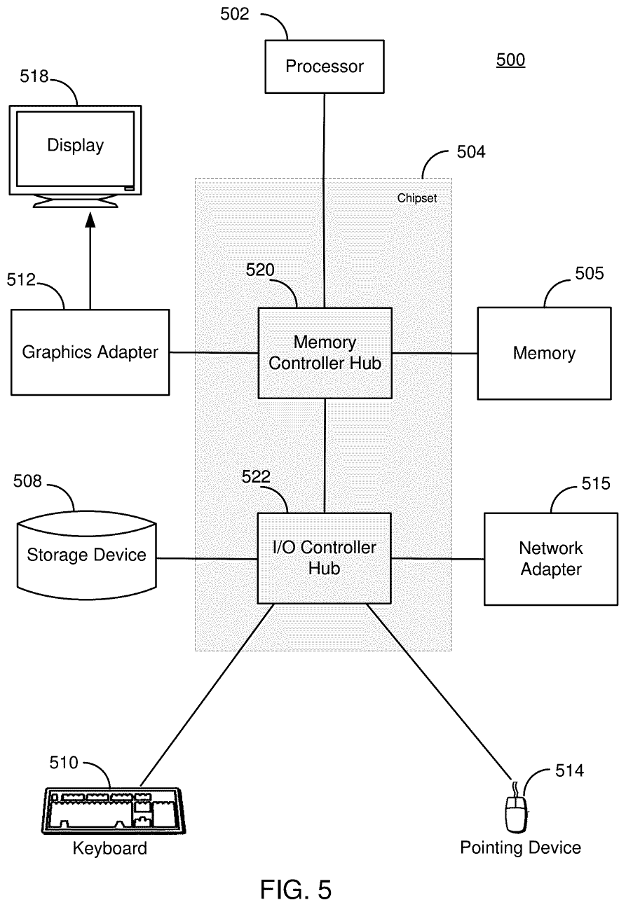

FIG. 5 is a block diagram of an example computer 500 suitable for use in analyzing the biological factors behind a vaccination reaction. The example computer 500 includes at least one processor 502 coupled to a chipset 504. The chipset 504 includes a memory controller hub 520 and an input/output (I/O) controller hub 522. A memory 506 and a graphics adapter 512 are coupled to the memory controller hub 520, and a display 518 is coupled to the graphics adapter 512. A storage device 508, keyboard 510, pointing device 514, and network adapter 516 are coupled to the I/O controller hub 522. Other embodiments of the computer 500 have different architectures.

In the embodiment shown in FIG. 5, the storage device 508 is a non-transitory computer-readable storage medium such as a hard drive, compact disk read-only memory (CD-ROM), DVD, or a solid-state memory device. The memory 506 holds instructions and data used by the processor 502. The pointing device 514 is a mouse, track ball, touch-screen, or other type of pointing device, and may be used in combination with the keyboard 510 (which may be an on-screen keyboard) to input data into the computer system 500. The graphics adapter 512 displays images and other information on the display 518. The network adapter 516 couples the computer system 500 to one or more computer networks, such as network 170.

The types of computers used by the entities of FIGS. 1 and 2 can vary depending upon the embodiment and the processing power required by the entity. For example, computing server 110 might include multiple blade servers working together to provide the functionality described. Furthermore, the computers can lack some of the components described above, such as keyboards 510, graphics adapters 512, and displays 518.

### Additional Considerations

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate+/−10% unless another meaning is apparent from the context. From example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs for a system and a process of determining biological pathways involved in a vaccination response. Thus, while particular embodiments and applications have been illustrated and described, it is to be understood that the described subject matter is not limited to the precise construction and components disclosed. The scope of protection should be limited only by the following claims.

What is claimed is:

1. A method comprising:
obtaining a dataset divided into layers, each layer including data of a corresponding data type, wherein at least some of the layers includes data values for a plurality of parameters corresponding to biological factors collected during different time periods;
selecting seed features from the plurality of parameters, wherein selected seed features are those known to be correlated to a phenotype;
constructing a seed correlation network indicating pairs of correlated seed features;
adding, to the seed correlation network, candidate nodes that are correlated to pairs of seed features;
identifying a plurality of paths in the seed correlation network between a pair of seed features;
determining a corresponding predictive power of a path of the plurality of paths, each corresponding predictive power indicating a likelihood of nodes in the path correctly predicting a vaccination response;
discarding one or more paths of the plurality of paths for which the corresponding predictive power is less than a predictive power of the pair of seed nodes; and
ranking each path in the plurality of paths based on the corresponding predictive power to provide insight into the biological factors.

2. The method of claim 1, wherein the layers are subsets of the dataset divided based on a time that a corresponding blood sample is taken.

3. The method of claim 1, wherein the seed features are correlated across multiple time points and significant edges are directed from a seed feature with an earlier timepoint to a seed feature with a later timepoint.

4. The method of claim 1, wherein the plurality of parameters include at least one of:
a protein layer,
an RNA layer,
an antibodies layer,
a white blood cell layer,
a red blood cell layer,
a cytokine layer, or
an adjuvant layer.

5. The method of claim 1, wherein selecting the seed features is based on a categorical question.

6. The method of claim 1, wherein the corresponding predictive power of the path is calculated based on a product of an area under a curve and an error rate associated with the path.

7. The method of claim 6, wherein the area under the curve and the error rate associated with the path is determined by fitting a random forest model to the path.

8. A system comprising one or more processors and one or more hardware storage devices having stored thereon computer-executable instructions that, when executed by the one or more processors, causes the computer system to:
obtain a dataset divided into layers, each layer including data of a corresponding data type, wherein at least some of the layers includes data values for a plurality of parameters corresponding to biological factors collected during different time periods;
select seed features from the plurality of parameters, wherein selected seed features are those known to be correlated to a phenotype;
construct a seed correlation network indicating pairs of correlated seed features;
add, to the seed correlation network, candidate nodes that are correlated to pairs of seed features;
identify a plurality of paths in the seed correlation network between a pair of seed features;
determine a corresponding predictive power of a path of the plurality of paths, each corresponding predictive power indicating a likelihood of nodes in the path correctly predicting a vaccination response;
discard one or more paths of the plurality of paths for which the corresponding predictive power is less than a predictive power of the pair of seed nodes; and
rank each path in the plurality of paths based on the corresponding predictive power to provide insight into the biological factors.

9. The system of claim 8, wherein the layers are subsets of the dataset divided based on a time that a corresponding tissue sample is taken.

10. The system of claim 8, wherein the seed features are correlated across multiple time points and significant edges are directed from a seed feature with an earlier timepoint to a seed feature with a later timepoint.

11. The system of claim 8, wherein the plurality of parameters include at least one of:
a protein layer,
an RNA layer,
an antibodies layer,
a white blood cell layer,
a red blood cell layer,
a cytokine layer, or
an adjuvant layer.

12. The system of claim 8, wherein selecting the seed features is based on a categorical question.

13. The system of claim 8, wherein the corresponding predictive power of the path is calculated based on a product of an area under a curve and an error rate associated with the path.

14. The system of claim 8, wherein an area under the curve and an error rate associated with the path is determined by fitting a random forest model to the path.

15. A non-transitory computer-readable medium configured to store code comprising instructions, wherein the instructions, when executed by one or more processors, cause the one or more processors to perform steps comprising:
obtaining a dataset divided into layers, each layer including data of a corresponding data type, wherein at least some of the layers includes data values for a plurality of parameters corresponding to biological factors collected during different time periods;
selecting seed features from the plurality of parameters, wherein selected seed features are those known to be correlated to a phenotype;
constructing a seed correlation network indicating pairs of correlated seed features;
adding, to the seed correlation network, candidate nodes that are correlated to pairs of seed features;
identifying a plurality of paths in the seed correlation network between a pair of seed features;
determining a corresponding predictive power of a path of the plurality of paths, each corresponding predictive power indicating a likelihood of nodes in the path correctly predicting a vaccination response;
discarding one or more paths of the plurality of paths for which the corresponding predictive power is less than a predictive power of the pair of seed nodes; and
ranking each path in the plurality of paths based on the corresponding predictive power to provide insight into the biological factors.

16. The non-transitory computer-readable medium of claim 15, wherein the layers are subsets of the dataset divided based on a time that a corresponding tissue sample is taken.

17. The non-transitory computer-readable medium of claim 15, wherein the seed features are correlated across multiple time points and significant edges are directed from a seed feature with an earlier timepoint to a seed feature with a later timepoint.

18. The non-transitory computer-readable medium of claim 15, wherein the plurality of parameters include at least one of:
a protein layer,
an RNA layer,
an antibodies layer,
a white blood cell layer,
a red blood cell layer,
a cytokine layer, or
an adjuvant layer.

19. The non-transitory computer-readable medium of claim 15, wherein the corresponding predictive power of the path is calculated based on a product of an area under a curve and an error rate associated with the path.

20. The non-transitory computer-readable medium of claim 15, wherein an area under the curve and an error rate associated with the path is determined by fitting a random forest model to the path.
